(19) United States
(12) Patent Application Publication
(10) Pub. No.: US 2023/0245765 A1
(43) Pub. Date: Aug. 3, 2023

(54) INFORMATION PROCESSING APPARATUS, INFORMATION PROCESSING METHOD, INFORMATION PROCESSING PROGRAM, AND STORAGE APPARATUS

(71) Applicant: FUJIFILM CORPORATION, Tokyo (JP)

(72) Inventors: Haruyasu NAKATSUGAWA, Kanagawa (JP); Yasuhisa KANEKO, Kanagawa (JP); Nobuya KITAMURA, Kanagawa (JP); Kenji NAGAMIYA, Kanagawa (JP); Tomohide HIRAGAMI, Kanagawa (JP); Yasuyuki HOSONO, Kanagawa (JP)

(21) Appl. No.: 18/298,373

(22) Filed: Apr. 11, 2023

Related U.S. Application Data
(63) Continuation of application No. PCT/JP2021/035991, filed on Sep. 29, 2021.

(30) Foreign Application Priority Data
Oct. 28, 2020 (JP) 2020-180638

(51) Int. Cl. G16H 40/20; G16H 15/00; G16H 50/80

(52) U.S. Cl. CPC G16H 40/20; G16H 15/00; G16H 50/80

(57) ABSTRACT

Provided is an information processing apparatus configured to: specify at least one of an object contacted by a person or an object approached by a person within a certain distance for a certain period of time or longer; and perform control to store log data for use in a notification of a cleaning request for an object in a storage apparatus, the log data including identification information of the specified object.

### CROSS-REFERENCE TO RELATED APPLICATIONS

This application is a continuation application of International Application No. PCT/JP2021/035991, filed on Sep. 29, 2021, which is incorporated herein by reference in its entirety. Further, this application claims priority from Japanese Patent Application No. 2020-180638, filed on Oct. 28, 2020, the disclosure of which is incorporated by reference herein in its entirety.

### BACKGROUND

### Technical Field

The present disclosure relates to an information processing apparatus, an information processing method, an information processing program, and a storage apparatus.

### Related Art

JP2015-533248A discloses that a memo that a person has approached a shelf is recorded together with identification information of the person who has approached the shelf in an emergency response environment.

WO2013/031481A discloses a technique for detecting at least one of contact or approach between objects.

JP2017-004165A discloses a technique for ascertaining infection routes through people and devices by extracting a device having a movement history that has approached a person who has been determined to have a disease, based on a person's movement history and a device's movement history, in a hospital.

### SUMMARY

In order to suppress the spread of infectious diseases, it is considered that it is useful to appropriately clean an object approached by a person and an object contacted by a person. However, in JP2015-533248A, WO2013/031481A, and JP2017-004165A, the cleaning of an object approached by a person and an object contacted by a person is not considered.

The present disclosure has been made in view of the above circumstances, and an object of the present disclosure is to provide an information processing apparatus, an information processing method, an information processing program, and a storage apparatus capable of suppressing the spread of an infectious disease through an object.

According to an aspect of the present disclosure, there is provided an information processing apparatus comprising at least one processor, in which the processor is configured to: specify at least one of an object contacted by a person or an object approached by a person within a certain distance for a certain period of time or longer; and perform control to store log data for use in a notification of a cleaning request for an object in a storage apparatus, the log data including identification information of the specified object.

In the information processing apparatus according to the aspect of the present disclosure, the processor may be configured to provide a notification of a cleaning request for an object whose identification information is included in the log data.

In the information processing apparatus according to the aspect of the present disclosure, the processor may be configured to provide the notification of the cleaning request at a timing predetermined as a timing at which the object is cleanable, for an area predetermined as a contamination controlled area in a hospital.

In the information processing apparatus according to the aspect of the present disclosure, the processor may be configured to provide the notification of the cleaning request at least one of a first timing or a second timing for areas other than an area predetermined as a contamination controlled area in a hospital, the first timing being when a person who has made at least one of contacting the object whose identification information is included in the log data or approaching the object within a certain distance for a certain period of time or longer and who has been located within the area is found to have a positive test result for an infectious disease, the second timing being when the person is found to satisfy a condition suspected of being infected with an infectious disease.

In the information processing apparatus according to the aspect of the present disclosure, the processor may be configured to provide the notification of the cleaning request for an object contacted by at least one person of the person who is found to have a positive test result for an infectious disease or the person who is found to satisfy a condition suspected of being infected with an infectious disease and for an object approached by the at least one person within a certain distance for a certain period of time or longer, during a period from at least one of the first timing or the second timing to before a second certain period of time.

In the information processing apparatus according to the aspect of the present disclosure, the second certain period of time may be determined for each material of an object according to a maximum residual period of a pathogen of an infectious disease.

In the information processing apparatus according to the aspect of the present disclosure, the processor may be configured to further provide, at least one of the first timing or the second timing, after a timing when at least one person of the person who is found to have a positive test result for an infectious disease or the person who is found to satisfy a condition suspected of being infected with an infectious disease has come into contact with the object whose identification information is included in the log data or the at least one person has approached the object within a certain distance for a certain period of time or longer, a notification of information indicating the person who has come into contact, within the second certain period of time from the timing, with the object that has not been cleaned.

In the information processing apparatus according to the aspect of the present disclosure, the processor may be configured to further provide a notification of a cleaning request for an object that has come into contact with an object that is a target of a cleaning request and has not been cleaned.

In the information processing apparatus according to the aspect of the present disclosure, the processor may be configured to provide the notification of the cleaning request with different messages at the first timing and the second timing.

In the information processing apparatus according to the aspect of the present disclosure, the processor may be configured to provide, in a case where the specified object is provided with a display device, a notification of a cleaning request to be displayed on the display device.

In the information processing apparatus according to the aspect of the present disclosure, the processor may be configured to delete, from among logs included in the log data, logs for which an incubation period of a pathogen of an infectious disease has elapsed from a time of acquisition of the logs.

In the information processing apparatus according to the aspect of the present disclosure, the processor may be configured to, in a case of specifying the object, vary the certain period of time according to a protection state of a person.

According to another aspect of the present disclosure, there is provided an information processing method executed by a processor included in an information processing apparatus, the information processing method comprising: specifying at least one of an object contacted by a person or an object approached by a person within a certain distance for a certain period of time or longer; and performing control to store log data for use in a notification of a cleaning request for an object in a storage apparatus, the log data including identification information of the specified object.

According to another aspect of the present disclosure, there is provided an information processing program for causing a processor included in an information processing apparatus to execute: specifying at least one of an object contacted by a person or an object approached by a person within a certain distance for a certain period of time or longer; and performing control to store log data for use in a notification of a cleaning request for an object in a storage apparatus, the log data including identification information of the specified object.

According to another aspect of the present disclosure, there is provided a storage apparatus incorporated in an information processing apparatus, in which the storage apparatus stores log data for providing a notification of a cleaning request for an object by the information processing apparatus, the log data including identification information of an object specified as at least one of an object contacted by a person or an object approached by a person within a certain distance for a certain period of time or longer for providing a notification of a cleaning request.

According to the aspects of the present disclosure, it is possible to suppress the spread of an infectious disease through an object.

### BRIEF DESCRIPTION OF THE DRAWINGS

FIG. 1 is a schematic configuration diagram of an information processing system.

FIG. 2 is a diagram showing an example of a log acquired by an acquisition apparatus.

FIG. 3 is a block diagram showing an example of a hardware configuration of an information processing apparatus.

FIG. 4 is a block diagram showing an example of a functional configuration of the information processing apparatus.

FIG. 5 is a diagram illustrating an example of a notification process of a cleaning request for an object at a first timing.

FIG. 6 is a flowchart showing an example of a log saving process.

FIG. 7 is a flowchart showing an example of a first cleaning request notification process.

FIG. 8 is a flowchart showing an example of a second cleaning request notification process.

FIG. 9 is a flowchart showing an example of a third cleaning request notification process.

FIG. 10 is a flowchart showing an example of a log deletion process.

FIG. 11 is a diagram illustrating an example of a notification process of a cleaning request for an object at a first timing.

FIG. 12 is a diagram illustrating an example of a notification process of a cleaning request for an object at a first timing.

### DETAILED DESCRIPTION

Hereinafter, form examples for implementing a technique of the present disclosure will be described in detail with reference to the drawings.

First, a configuration of an information processing system 10 according to the present embodiment will be described with reference to FIG. 1. As shown in FIG. 1, the information processing system 10 comprises an information processing apparatus 12 and a plurality of log acquisition apparatuses 14. The information processing apparatus 12 and each log acquisition apparatus 14 are each connected to a network so that they can communicate with each other. In the present embodiment, an example will be described in which the information processing apparatus 12 and each log acquisition apparatus 14 are installed in a hospital.

The log acquisition apparatus 14 acquires logs that detect that a person has come into contact with an object, that a person has left an object after coming into contact with the object, that a person has approached an object within a certain distance, and that a person has left an object for a certain distance or more after approaching the object within a certain distance. Examples of the person referred to here include a patient who visits a hospital, a family member of the patient, a medical worker who works at the hospital, and the like. In addition, examples of the object referred to here include an imaging device that captures a medical image, a control device that controls the imaging device, and an electrical device such as an operation terminal provided with a touch panel operated by a person. Note that, the object is not limited to the electrical device and may be an object other than the electrical device. Hereinafter, the fact that a person has approached an object within a certain distance is simply referred to as “approach”. In addition, the certain distance (for example, 10 cm) in this case may be predetermined, for example, according to the device used for detecting the approach, or may be predetermined according to the policy of the hospital.

Examples of the log acquisition apparatus 14 include an apparatus that detects an approach between a person and an object by short-range wireless communication such as an integrated circuit (IC) tag. In this case, a person is identified by individual identification information of the IC tag possessed by the person, and an object is identified by the individual identification information of an IC tag reader provided in the object. In addition, examples of the log acquisition apparatus 14 include an apparatus that detects contact between a person and an object by using electromagnetic noise generated by a smart watch worn on the arm of the person. In addition, an example of the log acquisition apparatus 14 is an apparatus that detects contact and approach between a person and an object by performing image analysis processing on a moving image captured by a camera at a predetermined frame rate. The log acquisition apparatus 14 is not limited to the above examples as long as it can detect that a person has come into contact with an object, that a person has left an object after coming into contact with the object, that a person has approached an object, and that a person has left an object after approaching the object, and may be an apparatus that performs the above detection based on other principles.

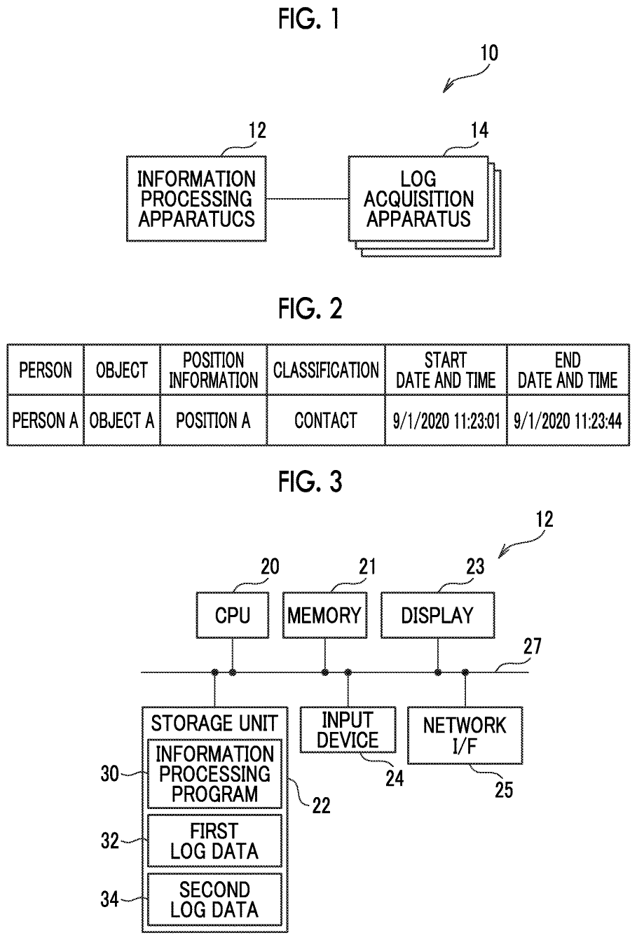

The log acquisition apparatus 14 transmits the acquired log to the information processing apparatus 12. FIG. 2 shows an example of a log. As shown in FIG. 2, the log acquired by the log acquisition apparatus 14 includes identification information of a person who has come into contact with or has approached an object, identification information of the object, position information indicating a position in a hospital where the object is installed, and a classification representing the contact or approach. In addition, as shown in FIG. 2, the log acquired by the log acquisition apparatus 14 further includes a start date and time, which is a date and time when a person starts contacting or approaching an object, and an end date and time of the contact or approach, which is a date and time when the person left an object after contacting or approaching the object.

Next, a hardware configuration of the information processing apparatus 12 according to the present embodiment will be described with reference to FIG. 3. As shown in FIG. 3, the information processing apparatus 12 includes a central processing unit (CPU) 20, a memory 21 as a temporary storage area, and a non-volatile storage unit 22. Further, the information processing apparatus 12 includes a display 23 such as a liquid crystal display, an input device 24 such as a keyboard and a mouse, and a network interface (I/F) 25 connected to a network. The CPU 20, the memory 21, the storage unit 22, the display 23, the input device 24, and the network I/F 25 are connected to a bus 27.

The storage unit 22 is realized by a hard disk drive (HDD), a solid state drive (SSD), a flash memory, or the like. An information processing program 30 is stored in the storage unit 22 as the storage medium. The CPU 20 reads out the information processing program 30 from the storage unit 22, loads the read-out program into the memory 21, and executes the loaded information processing program 30.

In addition, the storage unit 22 stores first log data 32 and second log data 34. The first log data 32 and the second log data 34 are log data for use in a notification of a cleaning request for an object. Specifically, the first log data 32 includes a log relating to the approach and contact of a person in a first area predetermined as a contamination controlled area in the hospital, and the second log data includes a log relating to the approach and contact of a person in a second area other than the first area in the hospital. Details of the first log data 32 and the second log data 34 will be described later. For example, the concept of zoning in hospitals can be applied to the division between the first area and the second area in the hospital.

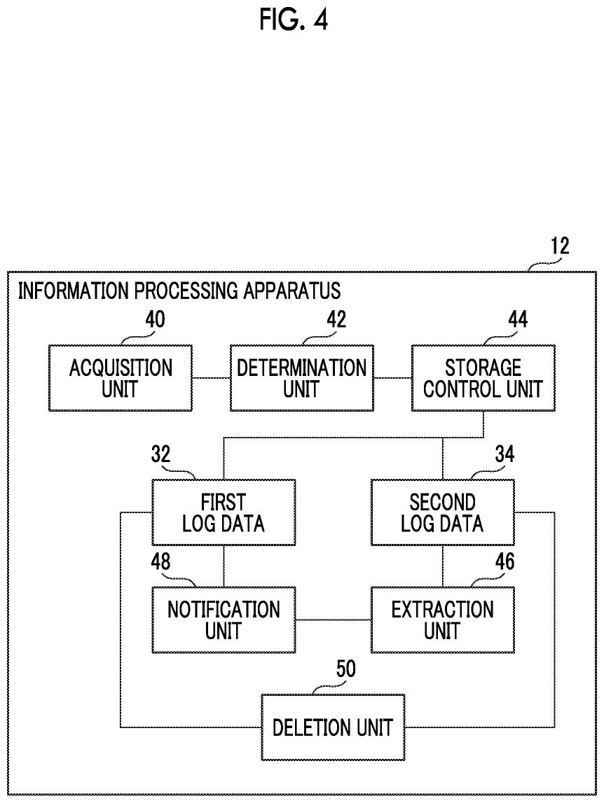

Next, a functional configuration of the information processing apparatus 12 according to the present embodiment will be described with reference to FIG. 4. As shown in FIG. 4, the information processing apparatus 12 includes an acquisition unit 40, a determination unit 42, a storage control unit 44, an extraction unit 46, a notification unit 48, and a deletion unit 50. The CPU 20 executes the information processing program 30 to function as the acquisition unit 40, the determination unit 42, the storage control unit 44, the extraction unit 46, the notification unit 48, and the deletion unit 50.

The acquisition unit 40 acquires the log transmitted from the log acquisition apparatus 14 via the network I/F 25.

The determination unit 42 uses the position information included in the log acquired by the acquisition unit 40, and determines whether or not an installation position of the object indicated by the identification information of the object included in the log is within the first area or the second area. In addition, the determination unit 42 determines whether or not the period from the “start date and time” to the “end date and time” is equal to or longer than a first certain period of time (for example, 15 minutes) for the log classified as “approach” among the logs acquired by the acquisition unit 40. The first certain period of time in this case may be predetermined, for example, according to the definition of close contact, or may be predetermined according to the policy of the hospital.

The storage control unit 44 performs control to store a log acquired by the acquisition unit 40 in the first log data 32 of the storage unit 22, the log being classified as “contact” and determined by the determination unit 42 that the installation position of the object is within the first area. In addition, the storage control unit 44 performs control to store a log acquired by the acquisition unit 40 in the second log data 34 of the storage unit 22, the log being classified as “contact” and determined by the determination unit 42 that the installation position of the object is within the second area. That is, a log including identification information of an object specified as having been contacted by a person in the first area is accumulated in the first log data 32. In addition, a log including identification information of an object specified as having been contacted by a person in the second area is accumulated in the second log data 34.

In addition, the storage control unit 44 performs control to store a log acquired by the acquisition unit 40 in the first log data 32 of the storage unit 22, the log being classified as “approach”, determined by the determination unit 42 that the installation position of the object is within the first area, and determined by the determination unit 42 that the period from the “start date and time” to the “end date and time” is equal to or longer than the first certain period of time. In addition, the storage control unit 44 performs control to store a log acquired by the acquisition unit 40 in the second log data 34 of the storage unit 22, the log being classified as “approach”, determined by the determination unit 42 that the installation position of the object is within the second area, and determined by the determination unit 42 that the period from the “start date and time” to the “end date and time” is equal to or longer than the first certain period of time. That is, a log including identification information of an object specified in the first area that a person has approached within a certain distance for a first certain period of time or longer is accumulated in the first log data 32. In addition, a log including identification information of an object specified in the second area that a person has approached within a certain distance for a first certain period of time or longer is accumulated in the second log data 34. In addition, the log classified as “approach” and determined by the determination unit 42 that the period from the “start date and time” to the “end date and time” is less than a first certain period of time is discarded among the logs acquired by the acquisition unit 40. Also, instead of discarding this log, the log may be included in the first log data 32 or the second log data 34 according to the installation position of the object. In this case, in the first log data 32 and the second log data 34, among the logs classified as “approach”, a notification flag may be set only for logs determined by the determination unit 42 that the period from the “start date and time” to the “end date and time” is equal to or longer than the first certain period of time.

The extraction unit 46 extracts, from the second log data 34, identification information of an object contacted by a person who is found to have a positive test result for an infectious disease at a first timing when a person whose identification information is included in the second log data 34 is found to have a positive test result for an infectious disease. A person whose identification information is included in the second log data 34 refers to a person who has made at least one of contacting an object whose identification information is included in the second log data 34 or approaching the object within a certain distance, and who has been located within the second area.

In addition, the extraction unit 46 also extracts, from the second log data 34, identification information of an object approached by a person who is found to have a positive test result for an infectious disease at the first timing. For example, the first timing is a timing at which the identification information of a person whose test result of the infectious disease is positive is input to the information processing apparatus 12.

In addition, the extraction unit 46 extracts, from the second log data 34, identification information of an object contacted by a person who is found to satisfy a condition suspected of being infected with an infectious disease at a second timing when a person whose identification information is included in the second log data 34 is found to satisfy a condition suspected of being infected with an infectious disease. A condition suspected of being infected with an infectious disease is, for example, a predetermined condition such as a body temperature of 37.5 degrees or higher or coughing.

In addition, the extraction unit 46 also extracts, from the second log data 34, identification information of an object approached by a person who is found to satisfy a condition suspected of being infected with an infectious disease at the second timing. For example, the second timing is a timing at which the identification information of a person who is found to satisfy a condition suspected of being infected with an infectious disease is input to the information processing apparatus 12.

The notification unit 48 provides a notification of a cleaning request for an object whose identification information is included in the first log data 32 at a timing predetermined as a timing at which the object can be cleaned for the first area. Examples of this timing include a break time such as a lunch break and a timing such as intervals between examinations.

In addition, the notification unit 48 provides a notification of a cleaning request for an object indicated by the identification information extracted by the extraction unit 46 at the first timing and the second timing for the second area. At this time, the notification unit 48 provides a notification of a cleaning request for an object contacted by the person who is found to have a positive test result for an infectious disease and for an object approached by the person for a first certain period of time or longer, which have not been cleaned, during a period from the first timing to before a second certain period of time. In addition, at this time, the notification unit 48 provides a notification of a cleaning request for an object contacted by the person who is found to satisfy a condition suspected of being infected with an infectious disease and for an object approached by the person for the first certain period of time or longer, which have not been cleaned, during a period from the second timing to before the second certain period of time. As the second certain period of time in this case, a period determined according to the maximum residual period of a pathogen of an infectious disease can be applied for each material of the object. For example, in a case where COVID-19 as an infectious disease is assumed, the maximum residual period of a virus as a pathogen is said to be 3 hours on a copper surface and 72 hours on a plastic surface. In this way, the second certain period of time may be set as the maximum residual period of the pathogen of the infectious disease for each material of the object, or may be set as a period in which a margin is added to the maximum residual period of the pathogen of the infectious disease. Further, in a case where a plurality of materials are used for the object, the second certain period of time may be set according to the material having the longest maximum residual period among the plurality of materials.

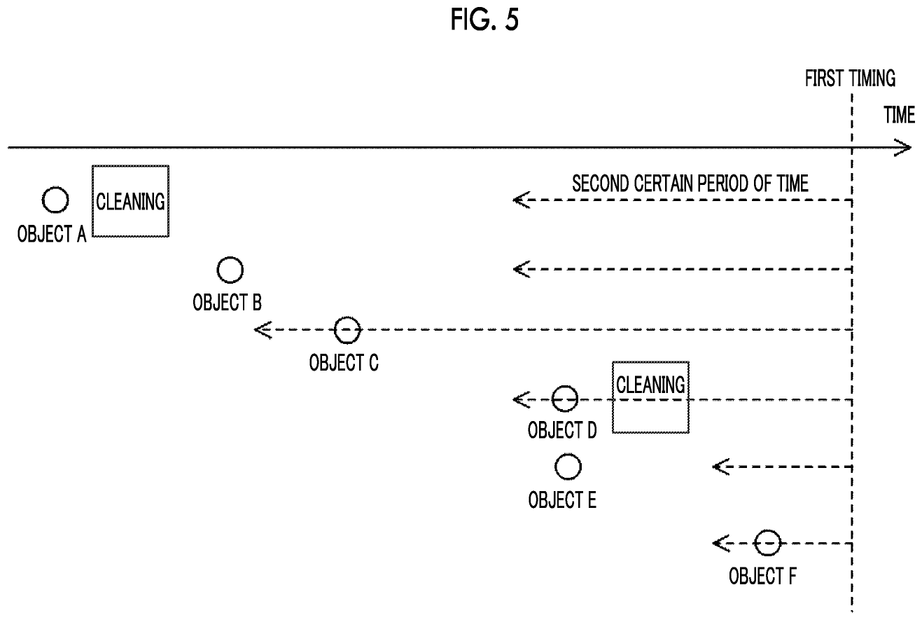

An example of a notification process of the cleaning request for an object at the first timing will be described with reference to FIG. 5. In FIG. 5, the horizontal axis represents time, and the circles represent an object whose identification information is included in the second log data 34 and a timing when a person who is found to have a positive test result for an infectious disease has come into contact with the object or the person has approached the object for a first certain period of time or longer. In addition, the leftward dashed arrow in FIG. 5 represents a second certain period of time. Further, FIG. 5 also shows the timing at which cleaning is performed on the object that has been cleaned.

In the example of FIG. 5, for an object A, an object B, and an object E, a notification of a cleaning request is not provided because a timing when a person who is found to have a positive test result for an infectious disease has come into contact with a corresponding object or the person has been approached a corresponding object for the first certain period of time or longer is a period from the first timing to before the second certain period of time. In addition, for an object D, a notification of a cleaning request is not provided because a timing when a person who is found to have a positive test result for an infectious disease has come into contact with a corresponding object or has been approached a corresponding object for the first certain period of time or longer is within the second certain period of time from the first timing but cleaning is being performed after contact or approach. In addition, for an object C and an object F, a notification of a cleaning request is provided because a timing when a person who is found to have a positive test result for an infectious disease has come into contact with a corresponding object or has been approached a corresponding object for the first certain period of time or longer is within the second certain period of time from the first timing and cleaning has not been performed. Although FIG. 5 shows an example at the first timing, the same applies to the second timing. Whether or not the object has been cleaned can be discriminated, for example, by associating identification information of the object with a flag indicating whether or not the object has been cleaned.

In the present embodiment, the notification unit 48 provides a notification of a cleaning request by transmitting a cleaning request message to a mobile information terminal possessed by a person in charge predetermined as an administrator of the object. An example of this message includes, for example, a message such as “Please clean the object A installed at the place A”. In addition, the notification unit 48 may provide the notification of the cleaning request by transmitting the cleaning request e-mail to the e-mail address of the person in charge predetermined as the administrator of the object. In addition, the notification unit 48 may provide the notification of the cleaning request by blinking a light emitting diode (LED) lamp provided on the object. In addition, in a case where the object is provided with a display device, the notification unit 48 may provide the notification of the cleaning request to be displayed on the display device. In addition, a plurality of these notification methods may be combined.

The deletion unit 50 deletes logs for which an upper limit value of an incubation period of the pathogen of the infectious disease has elapsed from the time of acquisition of the logs among the logs included in the first log data 32 and the second log data 34 at a periodic timing such as once a day. In a case where a plurality of infectious diseases are assumed, the deletion unit 50 deletes the log for which the longest incubation period of the incubation periods of the infectious diseases has elapsed from the time of acquisition of the logs among the logs included in the first log data 32 and the second log data 34. In addition, the deletion unit 50 may delete the log for which a period in which a margin is added to the incubation period of the pathogen of the infectious disease has elapsed from the time of acquisition of the logs.

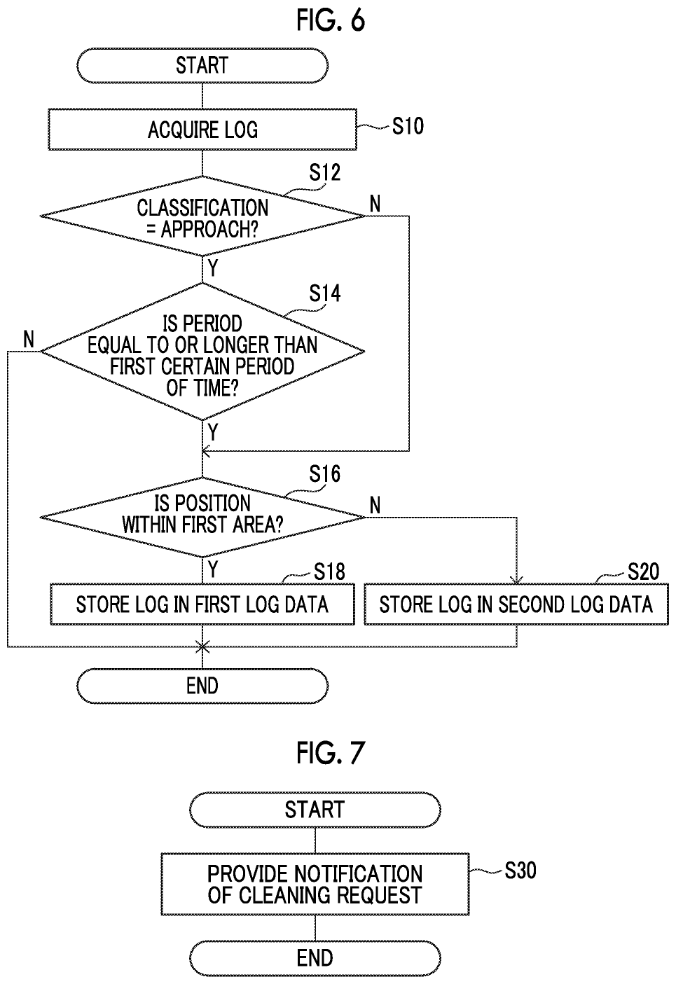

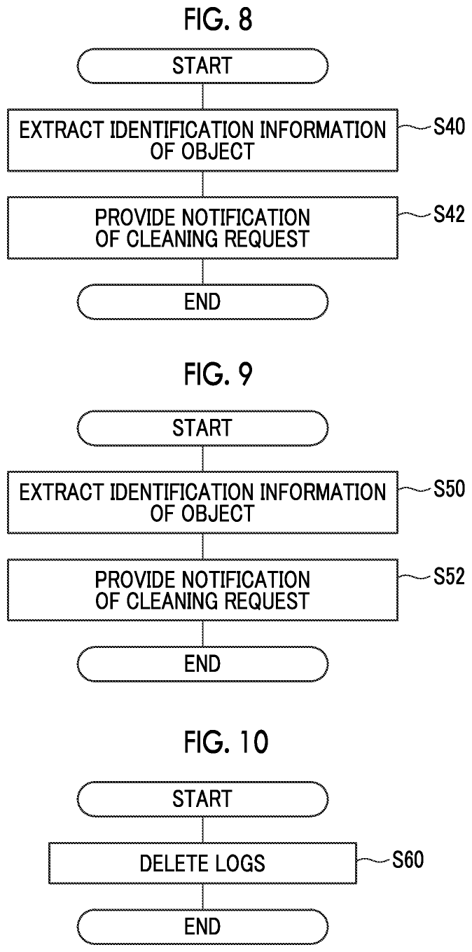

Next, operations of the information processing apparatus 12 according to the present embodiment will be described with reference to FIGS. 6 to 10. By executing the information processing program 30 by the CPU 20, a log saving process shown in FIG. 6, a first cleaning request notification process shown in FIG. 7, a second cleaning request notification process shown in FIG. 8, and a third cleaning request notification process shown in FIG. 9, and a log deletion process shown in FIG. 10 are executed. The log saving process shown in FIG. 6 is executed, for example, each time the information processing apparatus 12 receives a log transmitted from the log acquisition apparatus 14. In addition, the first cleaning request notification process shown in FIG. 7 is executed at a timing predetermined as a timing at which the object can be cleaned, for example, during a lunch break or intervals between examinations. Also, the second cleaning request notification process shown in FIG. 8 is executed, for example, at the first timing described above. Also, the third cleaning request notification process shown in FIG. 9 is executed, for example, at the second timing described above. The log deletion process shown in FIG. 10 is executed at a periodic timing such as once a day, for example.

In Step S10 of FIG. 6, the acquisition unit 40 acquires the log transmitted from the log acquisition apparatus 14 via the network I/F 25. In Step S12, the determination unit 42 determines whether or not the classification of the log acquired in Step S10 is “approach”. In a case where this determination is affirmative, the process proceeds to Step S14.

In Step S14, the determination unit 42 determines whether or not the period from the “start date and time” to the “end date and time” of the log acquired in Step S10 is equal to or longer than the first certain period of time. In a case where this determination is affirmative, the process proceeds to Step S16. In addition, in a case where the determination in Step S12 is negative, the process proceeds to Step S16.

In Step S16, the determination unit 42 uses the position information included in the log acquired in Step S10, and determines whether or not an installation position of the object indicated by the identification information of the object included in the log is within the first area. In a case where this determination is affirmative, the process proceeds to Step S18. In Step S18, the storage control unit 44 performs control to store the log acquired in Step S10 in the first log data 32 of the storage unit 22. In a case where the process of Step S18 is completed, the log saving process is completed.

On the other hand, in a case where the determination in Step S16 is negative, the process proceeds to Step S20. In Step S20, the storage control unit 44 performs control to store the log acquired in Step S10 in the second log data 34 of the storage unit 22. In a case where the process of Step S20 is completed, the log saving process is completed. In addition, in a case where the determination in Step S14 is negative, the log saving process is completed.

In Step S30 of FIG. 7, as described above, the notification unit 48 provides a notification of a cleaning request for the object whose identification information is included in the first log data 32. In a case where the process of Step S30 is completed, the first cleaning request notification process is completed.

In Step S40 of FIG. 8, the extraction unit 46 extracts, from the second log data 34, identification information of an object contacted by a person who is found to have a positive test result for an infectious disease. Further, the extraction unit 46 also extracts, from the second log data 34, identification information of an object approached by a person who is found to have a positive test result for an infectious disease. In Step S42, as described above, the extraction unit 46 provides a notification of a cleaning request for the object indicated by the identification information extracted in Step S40. In a case where the process of Step S42 is completed, the second cleaning request notification process is completed.

In Step S50 of FIG. 9, the extraction unit 46 extracts, from the second log data 34, identification information of an object contacted by a person who is found to satisfy a condition suspected of being infected with an infectious disease. Further, the extraction unit 46 also extracts, from the second log data 34, identification information of an object approached by a person who is found to satisfy a condition suspected of being infected with an infectious disease. In Step S52, as described above, the extraction unit 46 provides a notification of a cleaning request for the object indicated by the identification information extracted in Step S50. In a case where the process of Step S52 is completed, the third cleaning request notification process is completed.

In Step S60 of FIG. 10, as described above, the deletion unit 50 deletes logs for which an upper limit value of an incubation period of the pathogen of the infectious disease has elapsed from the time of acquisition of the logs among the logs included in the first log data 32 and the second log data 34. In a case where the process of Step S60 is completed, the log deletion process is completed.

As described above, according to the present embodiment, since the notification of the cleaning request for the object is appropriately provided, it is possible to suppress the spread of the infectious disease through the object.

In addition, in the above-described embodiment, the notification unit 48 may further provide, at the first timing, after a timing when the person who is found to have a positive test result for an infectious disease has come into contact with the object whose identification information is included in the log data 34 or the person has approached the object within a first certain period of time or longer, a notification of information indicating the person who has come into contact, within a second certain period of time from the timing, with the object that has not been cleaned. In this case, the notification unit 48 notifies, for example, a mobile information terminal possessed by the person of a message recommending disinfection, such as “You have come into contact with an object that may be contaminated, so please wash your hands”. In addition, the notification unit 48 may further provide, at the second timing, after a timing when the person who is found to have a positive test result for an infectious disease has come into contact with the object whose identification information is included in the log data 34 or the person has approached the object within a first certain period of time or longer, a notification of information indicating the person who has come into contact, within a second certain period of time from the timing, with the object that has not been cleaned.

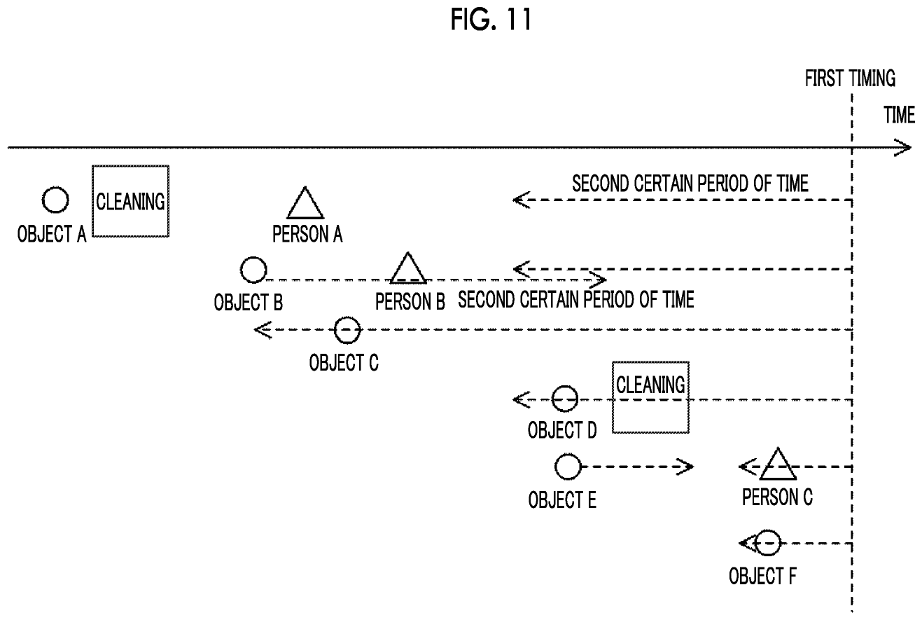

An example of a notification process of the cleaning request for an object at the first timing of this form example will be described with reference to FIG. 11. In FIG. 11, the horizontal axis represents time, and the circles represent an object whose identification information is included in the second log data 34 and a timing when a person who is found to have a positive test result for an infectious disease has come into contact with the object or the person has approached the object for a first certain period of time or longer. In addition, the leftward dashed arrow and the rightward dashed arrow in FIG. 11 represent a second certain period of time. Further, FIG. 11 also shows the timing at which cleaning is performed on the object that has been cleaned. Also, the triangular mark in FIG. 11 represents a person who has come into contact with the object in the same row. For example, a person A in FIG. 11 represents that the person A has come into contact with an object A after the object A has been cleaned.

In the example of FIG. 11, since the person A is in contact with the object A after the object A has been cleaned, the person A is not the target of the notification. In addition, since a person B is in contact with an object B within a second certain period of time after a person who is found to have a positive test result for an infectious disease has come into contact with the object B or the person has approached the object B for a first certain period of time or longer, the person B is the target of notification. In addition, since a person C is in contact with an object E after a second certain period of time has elapsed since a person who is found to have a positive test result for an infectious disease has come into contact with the object E or the person has approached the object E for a first certain period of time or longer, the person C is not the target of notification. Although FIG. 11 shows an example at the first timing, the same applies to the second timing.

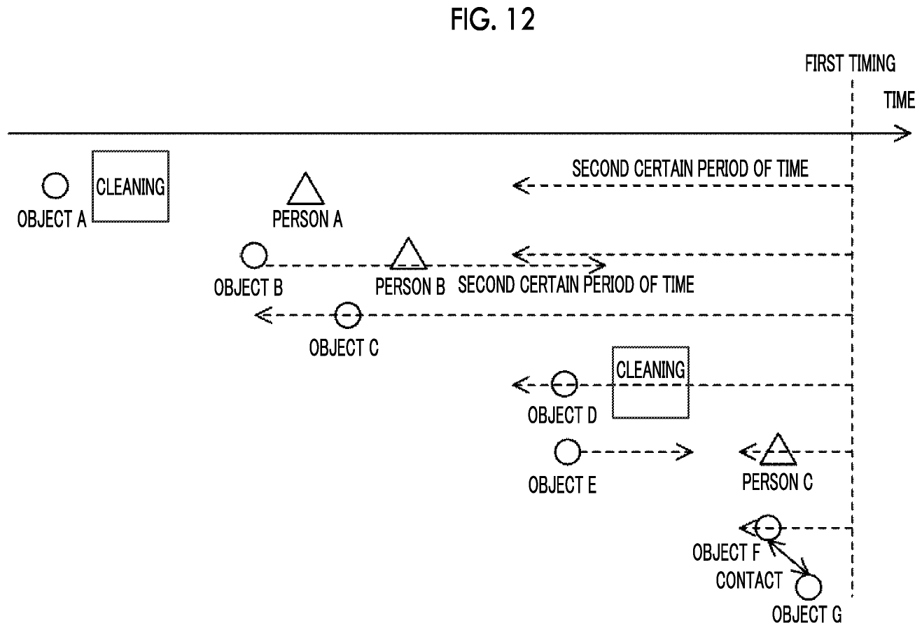

Further, in the above-described embodiment, the notification unit 48 may further provide, at the first timing and the second timing, a notification of a cleaning request for an object that has come into contact with an object that is the target of the cleaning request and has not been cleaned. In this form example, as shown in FIG. 12 as an example, a notification of a cleaning request for an object G that has come into contact with an object F that is the target of the cleaning request and has not been cleaned is also provided. Contact between the objects occurs, for example, in a situation where the objects are stored in a state where they are overlapped with each other.

In addition, in the above-described embodiment, the notification unit 48 may provide the notification of the cleaning request with different messages between the first timing and the second timing. In this case, the notification unit 48 provides, at the first timing, a notification of a cleaning request instructing cleaning, such as a message “It is an object contacted or approached by a person who has a positive test result for an infectious disease, so please clean it”, for example. On the other hand, in this case, the notification unit 48 provides, at the second timing, the notification of the cleaning request with a warning level lower than that of the first timing. Specifically, in this case, the notification unit 48 provides a notification of a cleaning request recommending cleaning, such as a message “It is an object contacted or approached by a person who is suspected of being infected with an infectious disease, so cleaning is recommended”, for example.

In addition, in the above-described embodiment, the determination unit 42 may vary the first certain period of time according to a protection state of a person. In this case, for example, in the determination unit 42, a form in which a person wearing a mask has a longer first certain period of time than a person not wearing a mask is exemplified. Whether or not a person wears a mask can be determined, for example, by performing image analysis processing on an image obtained by imaging the person with a camera.

In addition, in the above-described embodiment, the determination unit 42 may vary the first certain period of time according to a disinfection state of a person. In this case, for example, in the determination unit 42, a form in which a person who does not wash their hands sufficiently has a shorter first certain period of time than a person who washes their hands sufficiently is exemplified. Whether or not a person washes their hands sufficiently can be determined by, for example, a function of a wearable terminal such as a smart watch. For example, it is determined whether or not a person washes their hands sufficiently depending on whether or not the period of hand washing is equal to or greater than a predetermined threshold value.

Further, in the above-described embodiment, similarly to the log classified as “approach”, the log classified as “contact” may be controlled to store, in the storage unit 22, a log whose period from “start date and time” to “end date and time” is equal to or longer than a certain period of time. In this case, logs classified as “contact” and having a period from “start date and time” to “end date and time” shorter than a certain period of time are discarded. The certain period of time in this case may be the same as the first certain period of time or may be shorter than the first certain period of time.

In the above embodiments, for example, as hardware structures of processing units that execute various kinds of processing, such as the acquisition unit 40, the determination unit 42, the storage control unit 44, the extraction unit 46, the notification unit 48, and the deletion unit 50, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 30 is described as being stored (installed) in the storage unit 22 in advance; however, the present disclosure is not limited thereto. The information processing program 30 may be provided in a form recorded in a recording medium such as a compact disc read only memory (CD-ROM), a digital versatile disc read only memory (DVD-ROM), and a universal serial bus (USB) memory. In addition, the information processing program 30 may be downloaded from an external device via a network.

The disclosure of JP2020-180638 filed on Oct. 28, 2020 is incorporated herein by reference in its entirety. In addition, all literatures, patent applications, and technical standards described herein are incorporated by reference to the same extent as if the individual literature, patent applications, and technical standards were specifically and individually stated to be incorporated by reference.

What is claimed is:

1. An information processing apparatus comprising:
at least one processor,
wherein the processor is configured to:
specify at least one of an object contacted by a person or an object approached by a person within a certain distance for a certain period of time or longer; and
store log data for use in a notification of a cleaning request for an object in a memory, the log data including identification information of the specified object.

2. The information processing apparatus according to claim 1,
wherein the processor is configured to provide a notification of a cleaning request for an object whose identification information is included in the log data.

3. The information processing apparatus according to claim 2,
wherein the processor is configured to provide the notification of the cleaning request at a timing predetermined as a timing at which the object is cleanable, for an area predetermined as a contamination controlled area in a hospital.

4. The information processing apparatus according to claim 2,
wherein the processor is configured to provide the notification of the cleaning request at least one of a first timing or a second timing for areas other than an area predetermined as a contamination controlled area in a hospital, the first timing being when a person who has made at least one of contacting the object whose identification information is included in the log data or approaching the object within a certain distance for a certain period of time or longer and who has been located within the area is found to have a positive test result for an infectious disease, the second timing being when the person is found to satisfy a condition suspected of being infected with an infectious disease.

5. The information processing apparatus according to claim 4,
wherein the processor is configured to provide the notification of the cleaning request for an object contacted by at least one person of the person who is found to have a positive test result for an infectious disease or the person who is found to satisfy a condition suspected of being infected with an infectious disease and for an object approached by the at least one person within a certain distance for a certain period of time or longer, during a period from at least one of the first timing or the second timing to before a second certain period of time.

6. The information processing apparatus according to claim 5,
wherein the second certain period of time is determined for each material of an object according to a maximum residual period of a pathogen of an infectious disease.

7. The information processing apparatus according to claim 5,
wherein the processor is configured to further provide, at least one of the first timing or the second timing, after a timing when at least one person of the person who is found to have a positive test result for an infectious disease or the person who is found to satisfy a condition suspected of being infected with an infectious disease has come into contact with the object whose identification information is included in the log data or the at least one person has approached the object within a certain distance for a certain period of time or longer, a notification of information indicating the person who has come into contact, within the second certain period of time from the timing, with the object that has not been cleaned.

8. The information processing apparatus according to claim 4,
wherein the processor is configured to further provide a notification of a cleaning request for an object that has come into contact with an object that is a target of a cleaning request and has not been cleaned.

9. The information processing apparatus according to claim 4,
wherein the processor is configured to provide the notification of the cleaning request with different messages at the first timing and the second timing.

10. The information processing apparatus according to claim 1,
wherein the processor is configured to provide, in a case where the specified object is provided with a display, a notification of a cleaning request to be displayed on the display.

11. The information processing apparatus according to claim 1,
wherein the processor is configured to delete, from among logs included in the log data, logs for which an incubation period of a pathogen of an infectious disease has elapsed from a time of acquisition of the logs.

12. The information processing apparatus according to claim 1,
wherein the processor is configured to, in a case of specifying the object, vary the certain period of time according to a protection state of a person.

13. An information processing method comprising:
by a processor of an information processing apparatus,
specifying at least one of an object contacted by a person or an object approached by a person within a certain distance for a certain period of time or longer; and
storing log data for use in a notification of a cleaning request for an object in a storage apparatus, the log data including identification information of the specified object.

14. A non-transitory storage medium storing an information processing program for causing a processor included in an information processing apparatus to execute:
specifying at least one of an object contacted by a person or an object approached by a person within a certain distance for a certain period of time or longer; and
storing log data for use in a notification of a cleaning request for an object in a storage apparatus, the log data including identification information of the specified object.
